(19) United States
(12) Patent Application Publication
(10) Pub. No.: US 2022/0023137 A1
(43) Pub. Date: Jan. 27, 2022

(54) DEVICE AND METHOD FOR IMPROVING PERCEPTUAL ABILITY THROUGH SOUND CONTROL

(71) Applicant: DOKNIP BIOPHARMA, INC., Changwon-si, Gyeongsangnam-do (KR)

(72) Inventor: Hongwoon RHA, Seoul (KR)

(73) Assignee: DOKNIP BIOPHARMA, INC., Changwon-si, Gyeongsangnam-do (KR)

(21) Appl. No.: 17/297,609

(22) PCT Filed: Oct. 16, 2019
(86) PCT No.: PCT/KR2019/013604

§ 371 Date: May 27, 2021

(30) Foreign Application Priority Data
Nov. 28, 2018 (KR) 10-2018-0149990

(51) Int. Cl. A61H 5/00; G10K 15/02; G06F 3/16; H04S 7/00; H04R 3/04

(52) U.S. Cl. CPC A61H 5/00; G10K 15/02; H04R 25/75; H04S 7/30; H04R 3/04; G06F 3/165

(57) ABSTRACT

A device and a method for improving perceptual ability through sound control are disclosed. The device for improving perceptual ability includes a determination unit for determining a plurality of frequencies within a predetermined range from a frequency information and outputting a frequency-related information, and determining a reaction time between the plurality of frequencies and outputting a reaction time-related information; and a sound generation and output unit for rhythmizing a plurality of frequency signals on the basis of the frequency-related information and the reaction time-related information output from the determination unit to output a rhythmic auditory signal. The device can improve the hearing function.

### TECHNICAL FIELD

The present disclosure relates to a device and method for improving perceptual ability through a sound control, and more particularly, to a device and method for improving perceptual ability to improve and/or maintain an auditory or visual function through the sound control.

### BACKGROUND ART

The causes of hearing deterioration can be divided into an ear structure, an ear growth, and external factors. Among them, the main cause of hearing deterioration from 40 years old can be divided into deterioration due to exposure to ambient noise among the external factors or aging with age. Nowadays, children use smart phones and electronic devices a lot, so an environment is being built up where the children are exposed to noise. Among auditory organs including an outer ear, middle ear and inner ear, noise induced deafness due to this noise exposure and aging with age are significantly related to the cochlea of the inner ear. The problem is that this cochlear is accompanied by tinnitus in the sudden change area of some tonotopy frequencies with hearing deterioration.

A signal output method using the frequency characteristic according to human auditory characteristics and a tinnitus treatment device using the same are disclosed in Korean Patent Publication No. 10-0647310. In Korean Patent No. 10-0647310, a signal having a white noise stored in a memory for tinnitus treatment is output. However, stimulating only a specific frequency area with such a white noise may have some effect on tinnitus masking, but has a limit in terms of the training for tinnitus treatment. Most of all, the white noise may be recognized as the noise, so the stimulating may cause the rejection reaction.

In addition, a method and a device for stimulating auditory cells using an acoustic signal to improve hearing impairment are disclosed in Korean Patent Publication No. 10-0963888. In Korean Patent No. 10-0963888, user's auditory cells are stimulated by providing sound signals with resolutions of 1/k octave to determine a user's hearing threshold; determining a frequency of damaged auditory cells region on the basis of a determined hearing threshold; and outputting an acoustic signal having a predetermined intensity in a determined target frequency. However, intensive stimulation of only the frequency of specific auditory cells region may cause the generation of a deviation of the sensing ability between intensively stimulated auditory cells region and surrounding auditory cells region.

### DISCLOSURE

### Technical Problem

In order to improve the above-mentioned problem, an aspect of the present disclosure is to provide a device and method for improving perceptual ability that can improve auditory function by using a rhythmic sound without using an artificial noise.

In addition, another aspect of the present disclosure is to provide a device and method for improving perceptual ability that can improve and/or maintain auditory and visual functions by using the rhythmic sound got in consideration of auditory perception and/or cognition reaction times or the visual perception and/or cognitive reaction times.

### Technical Solution

According to one embodiment of the invention, a device for improving perceptual ability may comprise: a determination unit configured to determine a plurality of frequencies within a predetermined range from a frequency information and output a frequency-related information, and determine a reaction time between the plurality of frequencies and output a reaction time-related information; and a sound generation and output unit configured to rhythmize a plurality of frequency signals on the basis of the frequency-related information and the reaction time-related information output from the determination unit to output a rhythmic auditory signal.

According to a further embodiment of the invention, the determining unit may determine harmonic frequencies for each of the plurality of frequencies and output harmonic-related information, and the sound generation and output unit may synthesize harmonic frequency signals on the basis of the harmonic-related information output from the determining unit for each of the plurality of frequency signals and rhythmize a plurality of single tone frequency signals to output a rhythmic single tone frequency signal as the rhythmic auditory signal.

According to a further embodiment of the invention, the determining unit may determine at least one of brain waves as a period frequency and output a brain wave-related information, and the sound generation and output unit may convolute the rhythmic single tone frequency signal and an brain wave signal on the basis of the brain wave-related information output from the determining unit, to output the rhythmic auditory signal.

According to a further embodiment of the invention, the brain wave signal may be at least one of theta waves, alpha waves and beta waves.

According to a further embodiment of the invention, the determining unit may determine the plurality of frequencies using 2\hat{ }(M/L), wherein M represents the M^{th} musical scale with respect to a frequency, and L represents the number of musical scales within one octave range from the frequency.

According to a further embodiment of the invention, the determining unit may determine the plurality of frequencies from a band and sector table composed of frequencies according to piano music scales.

According to a further embodiment of the invention, the determining unit may sequentially match the plurality of frequencies with the reaction time-related information for each frequency-related information, in time order, to output a sequential frequency-reaction matching information, or randomly match the plurality of frequencies with the reaction time-related information for each frequency-related information to output a random frequency-reaction matching information; and the sound generation and output unit may rhythmize the plurality of frequency signals on the basis of the frequency-reaction matching information output from the determining unit to output the rhythmic auditory signal.

According to a further embodiment of the invention, the determination unit may determine an auditory reaction time of the sequential method within a range of 40 to 200 ms, and determine an auditory reaction time of the random method within the range of 20 to 160 ms.

According to a further embodiment of the invention, the sound generation and output unit may spatially process and output the rhythmic auditory signal in consideration of a visual reaction time.

According to a further embodiment of the invention, the sound generation and output unit may adjust a head-related transfer function (HRTF) so as to spatially process the rhythmic auditory signal.

According to a further embodiment of the invention, the sound generation and output unit may consider the visual reaction time within a range of 156 ms to 1.25 s.

According to a further embodiment of the invention, the sound generation and output unit may comprise multi-stage filters to perform a tuning function on the rhythmic auditory signal according to speaker terminal characteristics.

According to another embodiment of the invention, a method for improving perceptual ability may comprise: determining a plurality of frequencies within a predetermined range from a frequency information and outputting a frequency-related information; determining a reaction time between the plurality of frequencies and outputting a reaction time-related information; and rhythmizing a plurality of frequency signals on the basis of the frequency-related information and the reaction time-related information to output a rhythmic auditory signal.

### Advantageous Effects

According to the features described above, the present disclosure can improve an auditory function by using a rhythmic sound. That is, the rejection reaction can be reduced by not using an artificial noise, so continuous training or rehabilitation is possible.

In addition, the present disclosure can maximize the effect of improving hearing by using a single tone, such as sounds of musical instruments that user can concentrate on.

In addition, the present disclosure can provide a user with positive effects of brain wave by using a synchronized signal to brain wave as a period frequency.

In addition, the present disclosure can improve the visual function by inducing user's eye movement by spatially processing the rhythmic sound.

### DESCRIPTION OF DRAWINGS

The above and other aspects, features and advantages of the present disclosure will be more apparent from the following detailed description taken in conjunction with the accompanying drawings, in which:

FIG. 1 is a schematic block diagram of a device for improving perceptual ability according to an embodiment of the present disclosure;

FIG. 2 is a detailed block diagram of a device for improving perceptual ability according to another embodiment of the present disclosure;

FIG. 3 illustrates in detail a signal range determination part illustrated in FIG. 2;

FIG. 4 illustrates in detail a reaction time determination part illustrated in FIG. 2;

FIG. 5 illustrates in detail a sound generation part illustrated in FIG. 2;

FIG. 6 illustrates in detail a visual space process part illustrated in FIG. 2;

FIG. 7 illustrates in detail a sound output part illustrated in FIG. 2;

FIG. 8 illustrates the result of an audiogram before the device according to the present disclosure is used,

FIG. 9 illustrates the result of an audiogram after the device according to the present disclosure has been used for two months.

FIG. 10 is a flowchart illustrating a method for improving perceptual ability according to other embodiment of the present disclosure.

### MODE FOR INVENTION

Hereinafter, preferable embodiments of a device and a method for improving perceptual ability according to the present disclosure will be described with reference to the accompanying drawings. For reference, in the following description, the terms referring to elements of the present disclosure are denominated in consideration of the functions of the elements, and thus should not be construed to limit the technical elements of the present disclosure.

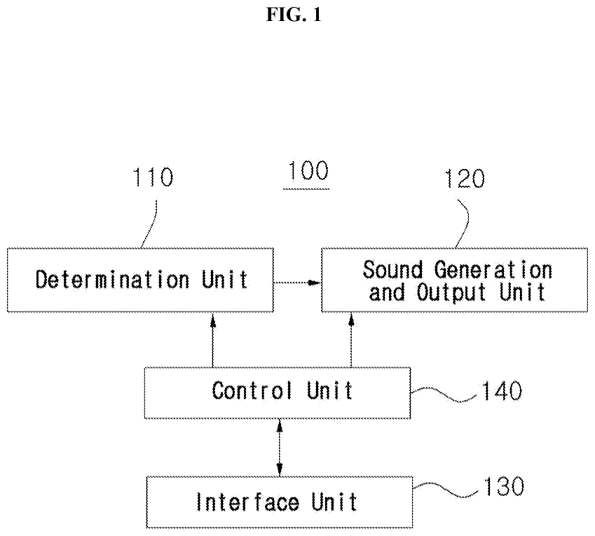

FIG. 1 is a schematic block diagram of a device for improving perceptual ability according to an embodiment of the present disclosure.

As shown in FIG. 1, a perceptual ability improving device 100 includes a determination unit 110, a sound generation and output unit 120, an interface unit 130, and a control unit 140.

The determination unit 110 may determine a plurality of frequencies within a predetermined range from each frequency information of a plurality of frequency informations and output a frequency-related information, and determine a reaction time between the plurality of frequencies and output a reaction time-related information. The determination unit 110 may also determine harmonic frequencies for each of the plurality of frequencies and output a harmonic-related information, and determine at least one of brain waves as a period frequency and output a brain wave-related information.

The sound generation and output unit 120 may rhythmize a plurality of frequency signals on the basis of the frequency-related information and the reaction time-related information output from the determination unit 110 to output a rhythmic auditory signal, for example, a rhythmic pure tone signal. The sound generation and output unit 120 may also synthesizes harmonic frequency signals on the basis of the harmonic-related information output from the determination unit 110 for each of the plurality of frequency signals and rhythmize a plurality of single tone frequency signals to output a rhythmic single tone frequency signal. The sound generation and output unit 120 may also convolute the rhythmic single tone frequency signal and a brain wave signal on the basis of the brain wave-related information output from the determination unit 110, to output the convoluted single tone frequency signal

The interface unit 130 is used for directly setting informations necessary for the determination unit 110 by a user or for receiving informations measured by using a special measuring equipment.

The control unit 140 may provide set informations or the like input through the interface unit 130 to the determination unit 110 or the sound generation and output unit 120, or control the determination unit 110 or the sound generation and output unit 120.

The frequency which the human body senses is analyzed to have a natural logarithmic (LN) distribution. However, it can be consistent with the result of an arithmetically weighted calculation with a Log 2 distribution, wherein the Log 2 distribution is the concept of the octave of the piano commonly used in relation to a harmonic frequency distribution. From now on, the equation is represented by log 2, i.e., 2\hat{ }(x/12).

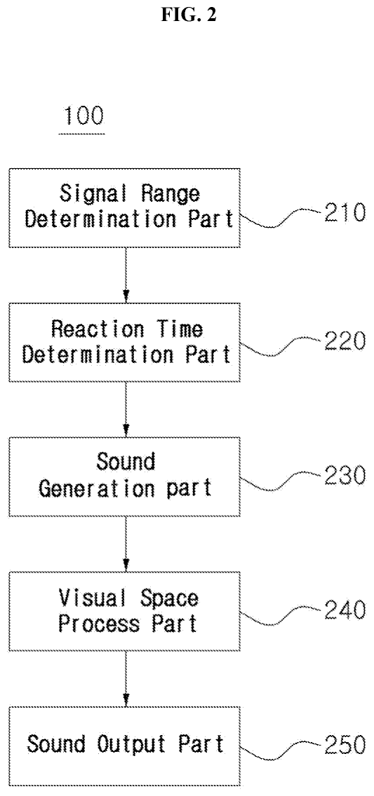

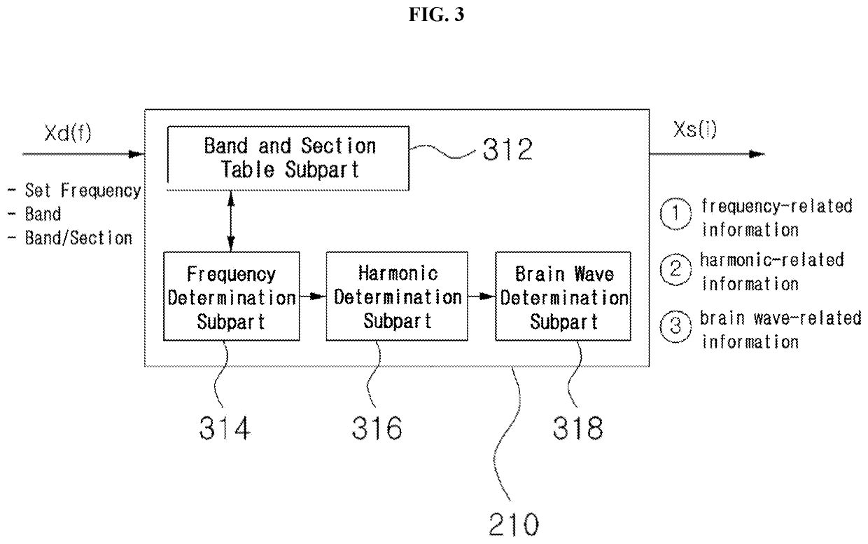

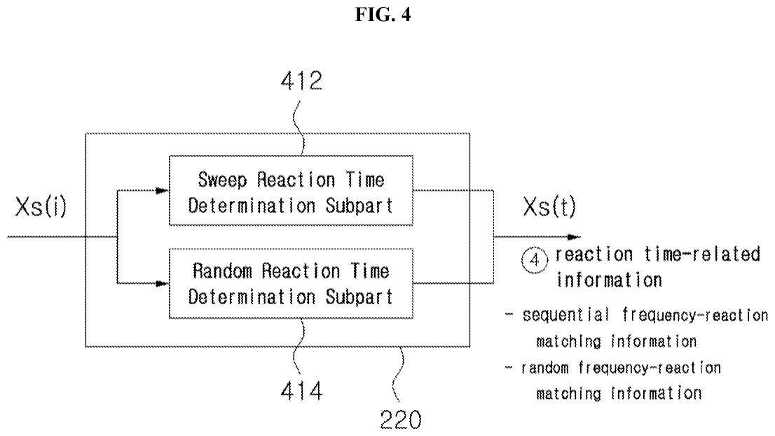

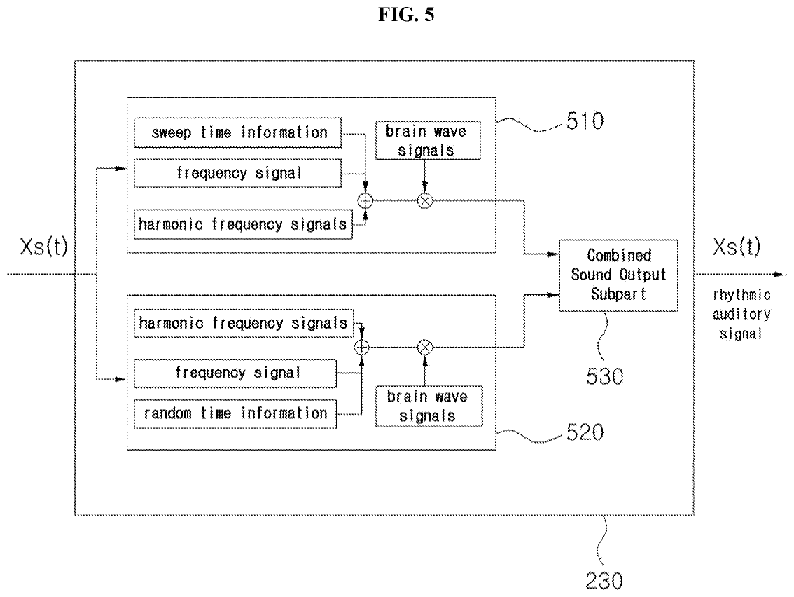

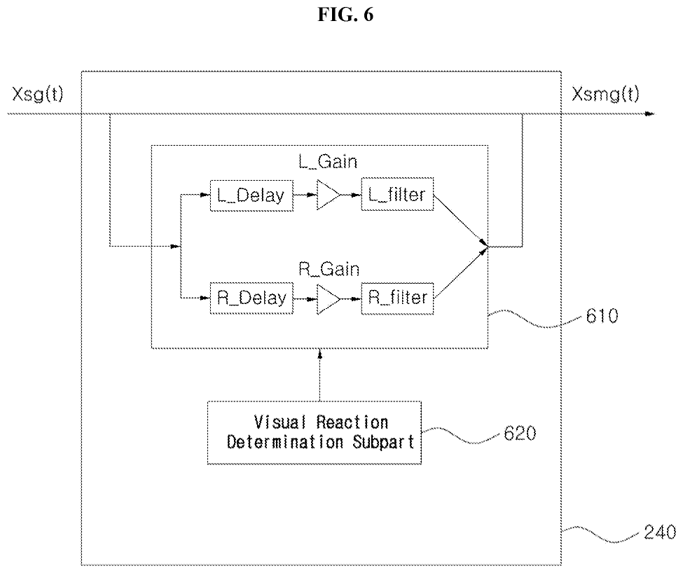

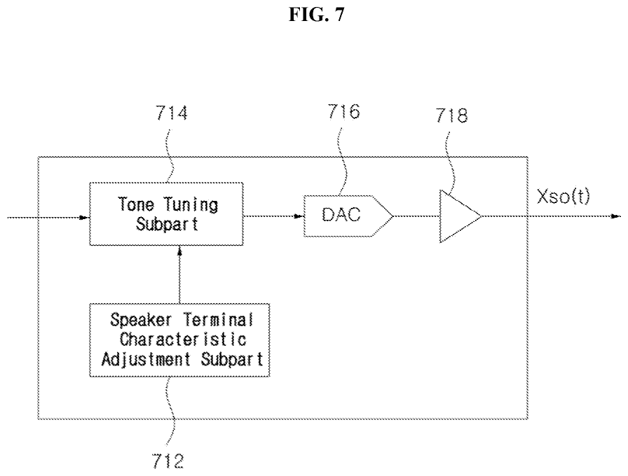

FIG. 2 is a detailed block diagram of a device for improving perceptual ability according to another embodiment of the present disclosure; FIG. 3 illustrates in detail a signal range determination part illustrated in FIG. 2; FIG. 4 illustrates in detail a reaction time determination part illustrated in FIG. 2; FIG. 5 illustrates in detail a sound generation part illustrated in FIG. 2; FIG. 6 illustrates in detail a visual space process part illustrated in FIG. 2; and FIG. 7 illustrates in detail a sound output part illustrated in FIG. 2.

As shown in FIG. 2, the perceptual ability improving device 100 may specifically include a signal range determination part 210, a reaction time determination part 220, a sound generation part 230, a visual space process part 240, and a sound output part 250.

When a frequency information specific to a user is set, the signal range determination part 210 determines a plurality of frequencies according to a set frequency information and outputs a frequency-related information. Here, the frequency information specific to the user may be a frequency set arbitrarily by the user, etc. or may be a frequency measured by using a specific measuring equipment, etc.

In the present disclosure, the plurality of frequencies that can be determined from the set frequency information can be calculated from the following [Equation 1]:

(A plurality of frequencies)={(set frequency)×2\hat{ }(M/L)} [Equation 1]

Here, M may be an integer representing the M^{th} musical scale from a set frequency. If M is a positive number, it maybe a musical scale in a direction higher than the set frequency (i.e., in the direction of high-pitched tone). If M is a negative number, it may be a musical scale in a direction lower than the set frequency (i.e., in the direction of low-pitched tone). L represents the number of musical scales within one octave range from the set frequency, and may be a natural number. The signal range determination part 210 illustrated in FIG. 2 is illustrated in detail in FIG. 3.

As illustrated in FIG. 3, the signal range determination part 210 may include a band and section table subpart 312, a frequency determination subpart 314, a harmonic determination subpart 316, and a brain wave determination subpart 318.

The band and section table subpart 312 may include a band and section table. In an embodiment of the present disclosure, each band may have a frequency range as illustrated in [Table 1]

TABLE 1

| Band Number (BN) | Frequency Range |
|---|---|
| B1 | 187 Hz~375 Hz |
| B2 | 250 Hz~500 Hz |
| B3 | 375 Hz~750 Hz |
| B4 | 500 Hz~1 kHz |
| B5 | 750 Hz~1.5 kHz |
| B6 | 1 kHz~2 kHz |
| B7 | 1.5 kHz~3 kHz |
| B8 | 2 kHz~4 kHz |
| B9 | 3 kHz~6 kHz |
| B10 | 4 kHz~8 kHz |
| B11 | 6 kHz~12 kHz |
| B12 | 8 kHz~16 kHz |

Further, each band may include a predetermined number of section frequencies calculated by Equation 1. For example, if the number of sections of each band is 12, frequencies based on the scale of a piano may be used. In this case, the range of each band corresponds to 1 octave of the piano, and 12 sectors correspond to 12 key boards per octave of the piano.

When the set frequency is provided, the frequency determination subpart 314 determines the closest frequency to the set frequency by using the band and section table as a reference frequency, and also determines a predetermined range of frequencies close to the reference frequency by using the band and section table as adjacent frequencies. The frequency determination subpart 314 may output information related to a plurality of determined frequencies, that is, the reference frequency and the adjacent frequencies, as frequency-related information. In this case, the frequency-related information output from the frequency determination subpart 314 may be an information related to a plurality of frequencies within the upper ½ octave to lower ½ octave range, that is, a 1 octave range in total, on the basis of the reference frequency.

In addition, when only a band table is provided, the frequency determination subpart 314 may determine frequencies of all 12 sections of the band designated by the user and the like as the plurality of frequencies. The frequency determination subpart 314 may output a related information of these frequencies as the frequency-related information.

Meanwhile, when the band and section table is provided, the frequency determination subpart 314 determines the section designated by the user as a reference frequency, and also determines a predetermined range of frequencies close to the reference frequency as adjacent frequencies, by using the band and section table. The frequency determination subpart 314 may output an information related to a plurality of determined frequencies, that is, the reference frequency and the adjacent frequencies, as the frequency-related information. In this case, the plurality of frequencies can be determined within the upper ¼ octave to lower ¼ octave range, that is, a ½ octave range in total, on the basis of the corresponding section.

The reference frequency-related information and the adjacent frequency-related information output from the frequency determination part 314 are informations related to pure tone frequencies. These frequency-related informations may include not only informations of the plurality of frequencies themselves but also informations obtained from the band and sector table.

The harmonic determination subpart 316 may determine harmonic frequencies for each of the plurality of frequencies, i.e., the reference frequency and the adjacent frequencies, determined by the frequency determination part 314 and output the harmonic-related information.

If the harmonic frequency is represented by Fhn, Fhn={(determined frequency)×n}. Here, when n is a positive number, the harmonic frequency is a multiple of the determined frequency, and when n is a negative number, the harmonic frequency is a fraction of the determined frequency. If n=2, 3, 4, and 5, the harmonic frequency is 2, 3, 4, and 5 times the determined frequency, respectively; if n=−2, −3, −4, and −5, the harmonic frequency is ½, ⅓, ¼, and ⅕ of the determined frequency, respectively.

The harmonic determination subpart 316 may determine the harmonic frequencies by using a plurality of harmonic information input through the interface unit 130. However, the information related to the harmonic frequencies may be stored in the harmonic determination subpart 316 as a default value.

The brain wave determination subpart 318 may determine a synchronized signal to a brain wave as a period frequency. In an embodiment of the present disclosure, theta waves, alpha waves, and beta waves may be used as the synchronized signal to the brain wave.

The signal range determination part 210 may output the frequency-related information, the harmonic-related information, and the brain wave-related information, as the informations output by the operation of the frequency determination subpart 314, the harmonic determination subpart 316, and the brain wave determination subpart 318, respectively.

The reaction time determination part 220 determines a reaction time between the plurality of frequency-related informations in consideration of auditory perception or cognition reaction time. The reaction time between the plurality of frequency-related informations may be one interval, but for example, the reaction time may be narrowed at a high frequency and may be widened at a low frequency, considering the frequency characteristics. In addition, the reaction time determination part 220 may output reaction times determined for each frequency-related information, as a reaction time-related information. The reaction time determination part 220 illustrated in FIG. 2 is illustrated in detail in FIG. 4.

The reaction time determination part 220 may include a sweep reaction time determination subpart 412 and a random reaction time determination subpart 414 in connection with the determination of the reaction time between the plurality of frequency-related informations.

The sweep reaction time determination subpart 412 determines a reaction time of a sweep method, wherein the sweep method sequentially outputs a plurality of frequencies, for example, in ascending or descending order, and the random reaction time determination subpart 414 determines a reaction time of a random method, wherein the random method outputs a plurality of frequencies in a random order which do not follow an ascending or descending order of frequencies or which output in any non-sequential order of reciprocating high and low pitched tones. The sweep method implemented in the present embodiment is intended for performing a massage-like function on the auditory cells, and the random method is intended for performing a pat-like function.

In relation to auditory sense, a time required in an intuitive reaction is about 5 ms, and a time required in a determination reaction is about 50 ms for the simple sound and about 150 to 200 ms for the complex sound. In the present exemplary embodiment, the reaction times of the sweep method and the random method may be set differently from each other in consideration of the auditory perception or cognition reaction time.

The sweep reaction time determination part 412 may be determined such that the frequencies of the sweep method are sequentially changed in the range of 40 to 200 ms, and the random reaction time determination part 414 may be determined such that the frequencies of the random method vary randomly in the range of 20 to 160 ms.

Here, for example, in the sweep method, the auditory reaction time may be determined to be greater than or equal to a minimum time wherein the minimum time is the time at which each sound which is sequentially output can be recognized to have been output, and/or may be determined to be less than or equal to a maximum time wherein the maximum time is the time at which each sound which is sequentially output will not be recognized to be cut off. In addition, in the random method, the auditory reaction time may be determined to be greater than or equal to a minimum recognition time and/or to be less than or equal to a maximum recognition time, wherein the minimum recognition time is time at which each sound which is randomly output can be recognized to have been output and wherein the maximum recognition time is the time at which the user can feel uncomfortable or the hearing improvement effect is insignificant.

The sound generation part 230 rhythmizes the plurality of frequency signals on the basis of the frequency-related information and the reaction time-related information, to output the rhythmic auditory signal. The sound generation unit 230 illustrated in FIG. 2 is illustrated in detail in FIG. 5.

The sound generation part 230 may include a sweep sound synthesis subpart 510, a random sound synthesis subpart 520, and a combined sound output subpart 530 so as to generate and output the rhythmic auditory signal.

The sweep sound synthesis subpart 510 may provide the corresponding frequency signal from the frequency-related information in the order of the sweep reaction time-related information determined by the sweep reaction time determination subpart 412; provide at least one corresponding harmonic frequency signal from the harmonic-related information; and add the at least one harmonic frequency signal to the frequency signal to generate a single tone sweep frequency signal. The sweep sound synthesis subpart 510 may also provide a corresponding brain wave signal from the brain wave-related information and convolute the single tone sweep frequency signal and the brain wave signal to output a convoluted single tone sweep auditory signal as the rhythmic auditory signal.

The random sound synthesis subpart 520 may provide a corresponding frequency signal from the frequency-related information in the order of the random time information determined by the random response time determination subpart 414; provide at least one corresponding harmonic frequency signal from the harmonic-related information; and add the at least one harmonic frequency signal to the frequency signal to generate a single tone random frequency signal. The random sound synthesis subpart 520 may provide the corresponding brain wave signal from the brain wave-related information, and convolute the signal tone random frequency signal and the bran wave signal to output a convoluted single tone random auditory signal as the rhythmic auditory signal.

Here, if the reference frequency of pure tone is Fp=A sin(t), the adjacent frequency of pure tone is Fs=A sin(t+ΔM), the harmonic frequency of the reference frequency is Fhn=Bn sin(t/n), and the harmonic frequency of the adjacent frequency is Fhns=Bn sin(t/n+ΔM), the array of pure tones is Fpsum={Fp, Σs} but the array of single tones is Fssum={(Fp+ΣFhn), Σ(Fs+ΣFhns)} (where the relationship of A with B is A>B).

The combined sound output subpart 530 may combine the sweep sound synthesis signal output from the sweep sound synthesis subpart 510 and the random sound synthesis signal output from the random sound synthesis subpart 520 to output a combined frequency signal.

The combined sound output subpart 530 may output the sound obtained by means of the sweep method and the sound obtained by means of the random method, alternately or in an arbitrary order. It is also possible to output the sound by inserting a blank time or output the sound continuously without a blank when alternating these methods.

The visual space process part 240 may spatially process and output the rhythmic auditory signal in consideration of visual perception or cognition reaction time. The visual space process part 240 illustrated in FIG. 2 is illustrated in detail in FIG. 6.

The visual space process part 240 may include a head-related transfer function (HRTF) adjustment subpart 610 for spatially processing the rhythmic auditory signal and a visual reaction determination subpart 620 for determining and providing a visual reaction time to the HRTF adjustment subpart 610.

The HRTF adjustment subpart 610 may include an L_Delay, an amplifier (L_Gain) and an L_Filter to process a left signal of the rhythmic auditory signal, and an R_Delay, an amplifier (R_Gain) and an R_Filter to process a right signal of the rhythmic auditory signal.

An L_Delay and an R_Delay delay each signal according to a set time, an L_Gain and an R_Gain adjust gains of each delayed signal, and each adjusted signal is filtered through an L_Filter and an R_Filter, respectively. The HRTF adjustment subpart 610 may adjust a position of a sound source of the rhythmic auditory signal left, right, front or back and adjust a range of a space, by these configurations.

When a still picture is displayed greater than or equal to 16 frames per second, a visual organ may feel a still picture as a moving picture. In this case, the time of one frame corresponds to 62.5 ms. Meanwhile, in the case of a simple ON/OFF display, when ON instruction is issued within 7.8 ms interval, the visual organ recognizes that the display is still ON. Therefore, in the case of the simple ON/OFF display, a reaction time of about 78 ms is required for an intuition reaction and a reaction time of at least 156 ms is required for a determination reaction. In the case of the complex image, a reaction time of about 625 ms is required for the intuition reaction and a reaction time of about 1.25 sec is required for the determination reaction. Therefore, the visual reaction time determination subpart 620 may determine the visual reaction time such that the visual reaction time is in the range of 156 ms to 1.25 s.

The sound output unit 250 may include multi-stage filters thus to perform a tuning function according to a speaker terminal characteristics. The sound output part 250 illustrated in FIG. 2 is illustrated in detail in FIG. 7.

The sound output unit 250 may include a speaker terminal characteristic adjustment subpart 712, a tone tuning subpart 714, a digital analog converter (DAC) 716, and an amplifier 718 so as to output an analog sound signal for improving the perceptual ability.

As the analog sound signal output from the sound output part 250 due to the speaker terminal characteristics may be distorted, the speaker terminal characteristic adjustment subpart 712 may adjust the characteristics of the tone tuning subpart 714 according to the speaker terminal characteristics in order to prevent such a distortion. The tone tuning subpart 714 may be configured by multi-stage filters (Filter1, Filter2 . . . FilterN) in preparation for the case that the reaction characteristics of the speaker terminal characteristics adjusted by the speaker terminal characteristic adjustment subpart 712 is different. The tone tuning subpart 714 may include a high pass filter (HPF), a low pass filter (LPF), a band pass filter (BPF), and the like so as to perform the tuning function of the speaker terminal. If no tuning is required, the tuning function may not be performed.

Through the final volume adjustment at the amplifier 718, the amplifier 718 may control and manage a gain so that the sound is less than an audible sound in the case of the training for improving hearing and adjust and manage the gain in the range of the audible sound in the case of improving eyesight. In addition, in the case of management involved in the brain wave tuning, the gain can be freely controlled.

The speaker terminal signal may be applied to earphones, headphones, sound pressure output devices, bone conduction speakers, and the like, to output sound to the user. In addition, a sound intended for enhancing hearing may also be created as sounds for left ear and right ear, respectively.

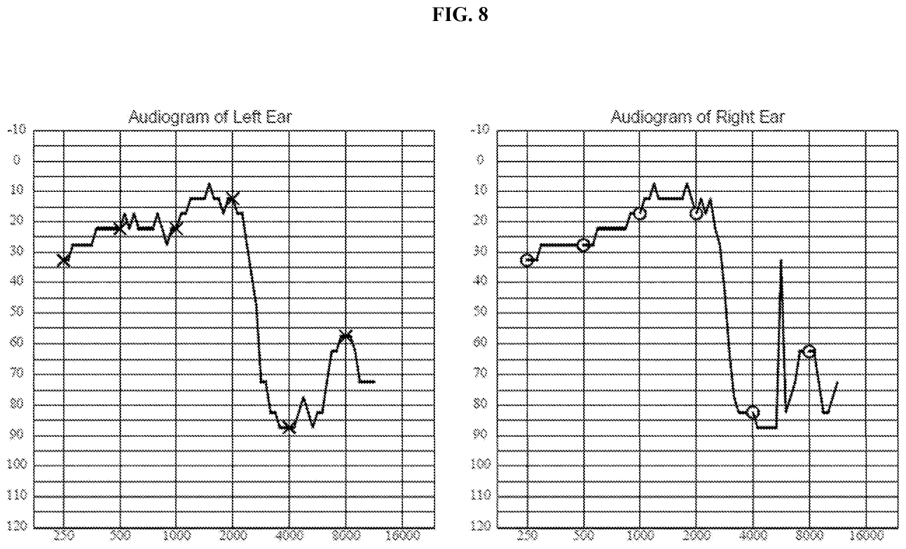

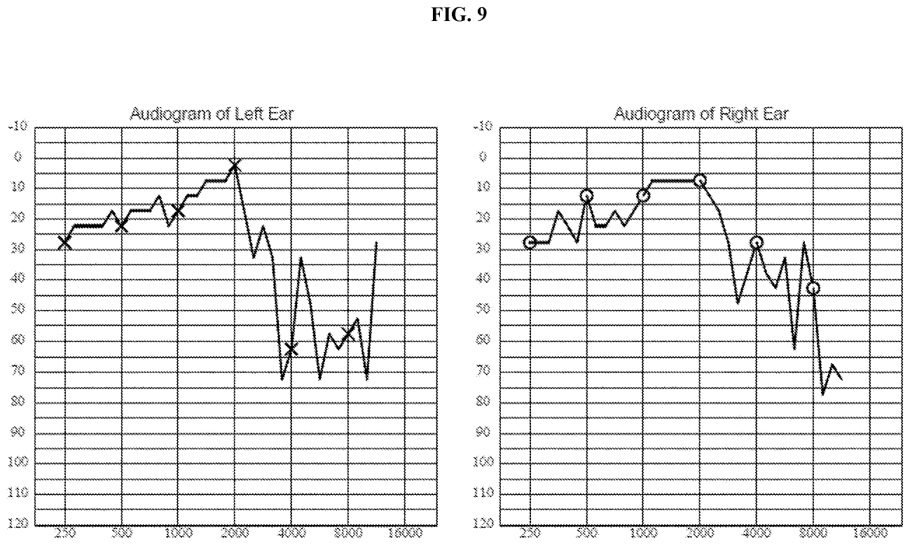

FIG. 8 illustrates the result of an audiogram before the device according to the present disclosure is used, and FIG. 9 illustrates the result of an audiogram after the device according to the present disclosure is used for two months.

Look at the 4 KHz band of FIG. 9, It can be seen that hearing is significantly improved after the use of the device according to the present disclosure.

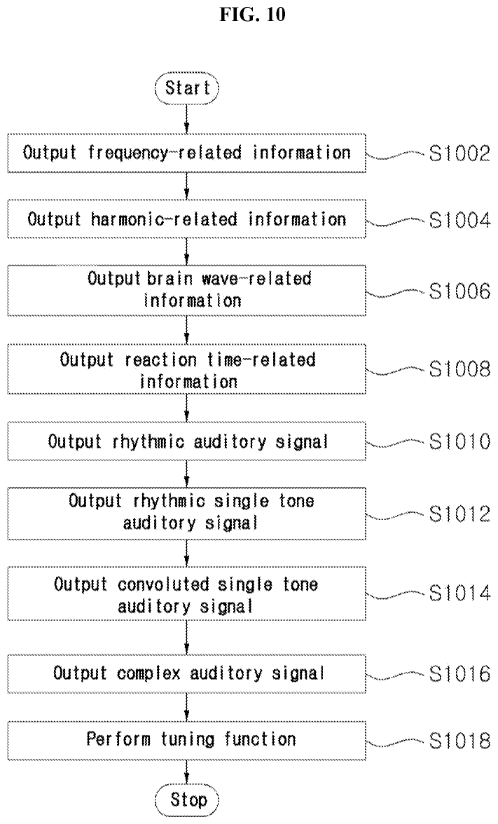

FIG. 10 is a flowchart illustrating a method for improving perceptual ability according to other embodiment of the present disclosure.

The determination unit 110 may determine a plurality of frequencies within a predetermined range from each frequency information of a plurality of frequency informations and output a frequency-related information (S1002). The determination unit 110 may also determine harmonic frequencies for each of the plurality of frequencies and output a harmonic-related information (S1004) and may determine at least one of brain waves as a period frequency and output a brain wave-related information (S1006).

The determination unit 110 may determine a reaction time between the plurality of frequencies and output a reaction time-related information (S1008). The determination unit 110 may sequentially match the plurality of frequencies with the reaction time-related information for each frequency-related information, in time order, to output a sequential frequency-reaction matching information, and may randomly match the plurality of frequencies with the reaction time-related information for each frequency-related information to output a random frequency-reaction matching information.

The sound generation and output unit 120 may rhythmize a plurality of frequency signals on the basis of the frequency-related information and the reaction time-related information output from the determination unit 110 and output a rhythmic auditory signal (S1010). When the frequency-reaction matching information output from the determination unit 110 is input, the sound generation and output unit 120 may rhythmize the plurality of frequency signals on the basis of the frequency-reaction matching information and output a rhythmic sweep auditory signal and a rhythmic random auditory signal.

The sound generation and output unit 120 may also synthesize harmonic frequency signals on the basis of the harmonic-related information output from the determination unit 110 for each of the plurality of frequency signals and rhythmize a plurality of single tone frequency signals to output a rhythmic single tone auditory signal (S1012). The sound generation and output unit 120 may convolute the rhythmic single tone frequency signal and the brain wave signal on the basis of the brain wave-related information output from the determination unit 110, to output a convoluted single tone auditory signal (S1014).

The sound generation and output unit 120 may spatially process the rhythmic auditory signal in consideration of a visual reaction time to output a complex auditory signal (S1016). The sound generation and output unit 120 may include multi-stage filters to perform a tuning function on the complex auditory signal according to speaker terminal characteristics (S1018).

The embodiments of the present disclosure are merely examples of the technical idea of the present disclosure, and the scope of the present disclosure should be interpreted based on the claims. Further, it can be understood by those skilled in the art that various modifications and changes can be made without departing from the essential features of the present disclosure and that all technical ideas within the equivalent range to the present disclosure should be construed as being included in the scope of the present disclosure.

1. A device for improving perceptual ability, the device comprising:
a determination unit configured to determine a plurality of frequencies within a predetermined range from a frequency information and output a frequency-related information, and determine a reaction time between the plurality of frequencies and output a reaction time-related information; and
a sound generation and output unit configured to rhythmize a plurality of frequency signals on the basis of the frequency-related information and the reaction time-related information output from the determination unit to output a rhythmic auditory signal.

2. The device of claim 1, wherein the determining unit is configured to determine harmonic frequencies for each of the plurality of frequencies and output harmonic-related information, and the sound generation and output unit is configured to synthesize harmonic frequency signals on the basis of the harmonic-related information output from the determining unit for each of the plurality of frequency signals, and rhythmize a plurality of single tone frequency signals to output a rhythmic single tone frequency signal as the rhythmic auditory signal.

3. The device of claim 2, wherein the determining unit is configured to determine at least one of brain waves as a period frequency and output a brain wave-related information, and the sound generation and output unit is configured to convolute the rhythmic single tone frequency signal and an brain wave signal on the basis of the brain wave-related information output from the determining unit, to output the rhythmic auditory signal.

4. The device of claim 3, wherein the brain wave signal is at least one of theta waves, alpha waves and beta waves.

5. The device of claim 1, wherein the determining unit is configured to determine the plurality of frequencies using 2\hat{ }(M/L), wherein M represents the Mth musical scale with respect to a frequency, and L represents the number of musical scales within one octave range from the frequency.

6. The device of claim 5, wherein the determining unit is configured to determine the plurality of frequencies from a band and sector table composed of frequencies according to piano music scales.

7. The device of claim 1, wherein the determining unit is configured to sequentially match the plurality of frequencies with the reaction time-related information for each frequency-related information, in time order, to output a sequential frequency-reaction matching information, or randomly match the plurality of frequencies with the reaction time-related information for each frequency-related information to output a random frequency-reaction matching information; and the sound generation and output unit is configured to rhythmize the plurality of frequency signals on the basis of the frequency-reaction matching information output from the determining unit to output the rhythmic auditory signal.

8. The device of claim 7, wherein the determination unit is configured to determine an auditory reaction time of the sequential method within a range of 40 to 200 ms, and determine an auditory reaction time of the random method within the range of 20 to 160 ms.

9. The device of claim 1, wherein the sound generation and output unit is configured to spatially process and output the rhythmic auditory signal in consideration of a visual reaction time.

10. The device of claim 9, wherein the sound generation and output unit is configured to adjust a head-related transfer function (HRTF) so as to spatially process the rhythmic auditory signal.

11. The device of claim 10, wherein the sound generation and output unit is configured to consider the visual reaction time within a range of 156 ms to 1.25 s.

12. The device of claim 1, wherein the sound generation and output unit comprises multi-stage filters to perform a tuning function on the rhythmic auditory signal according to speaker terminal characteristics.

13. A method for improving perceptual ability, the method comprising:
determining a plurality of frequencies within a predetermined range from a frequency information and outputting a frequency-related information;
determining a reaction time between the plurality of frequencies and outputting a reaction time-related information; and
rhythmizing a plurality of frequency signals on the basis of the frequency-related information and the reaction time-related information to output a rhythmic auditory signal.

14. The method of claim 13, wherein the outputting of the frequency-related information further comprises determining harmonic frequencies for each of the plurality of frequencies and outputting a harmonic-related information; and the outputting of the rhythmic auditory signal further comprises synthesizing harmonic frequency signals on the basis of the harmonic-related information for each of the plurality of frequency signals and rhythmizing a plurality of single tone frequency signals to output a rhythmic single tone frequency signal as the rhythmic auditory signal.

15. The method of claim 14, wherein the outputting of the frequency-related information further comprises determining at least one of brain waves and outputting a brain wave-related information as a period frequency; and the outputting of the rhythmic auditory signal further comprises convoluting the rhythmic single tone frequency signals and an a brain wave signal on the basis of the brain wave-related information, to output the rhythmic auditory signal.

16. The method of claim 13, wherein the outputting of the reaction time-related information further comprises sequentially matching the plurality of frequencies with the reaction time-related information for each frequency-related information, in time order, to output a sequential frequency-reaction matching information, or randomly matching the plurality of frequencies with the reaction time-related information for each frequency-related information to output a random frequency-reaction matching information; and the outputting of the rhythmic auditory signal further comprises rhythmizing the plurality of frequency signals on the basis of the frequency-reaction matching information to output the rhythmic auditory signal.

17. The method of claim 13, further comprising spatially processing the rhythmic auditory signal in consideration of visual reaction time to output a complex auditory signal.

18. The method of claim 17, further comprising performing a tuning function on the complex auditory signal according to speaker terminal characteristics, by using a multi-stage filter.
